(19) United States
(12) Patent Application Publication
(10) Pub. No.: US 2023/0233629 A1
(43) Pub. Date: Jul. 27, 2023

(54) METHOD TO TREAT HEPATOMA WITH DENGUE VIRUSES AND A METHOD TO KILL HEPATOCELLULAR CARCINOMA TISSUES

(71) Applicant: Syi Biotechnology Co., Ltd., Taichung City (TW)

(72) Inventors: Chia-Chang Chen, Taichung City (TW); Guey-Chuen Perng, Tainan City (TW); Hsiu-Man Lien, Taichung City (TW); Yi-Ju Chen, Taichung City (TW)

(21) Appl. No.: 17/871,085

(22) Filed: Jul. 22, 2022

(30) Foreign Application Priority Data
Jan. 27, 2022 (TW) 111103686

(51) Int. Cl. A61K 35/76; A61P 35/00; C12N 7/04

(52) U.S. Cl. CPC A61K 35/76; A61P 35/00; C12N 7/045; C12N 2770/24041

(57) ABSTRACT

A method to treat hepatoma with dengue viruses which infect liver tumor stem cells for annihilation of hepatocellular carcinoma tissues. The liver tumor stem cells expressing the biomarker of CD133 in a tumor part are the objects infected by dengue viruses preferentially and killed due to specific protein expressions for suppression of hepatoma.

### BACKGROUND OF THE INVENTION

### Field of the Invention

The present invention relates to a method to treat hepatoma with dengue viruses and particularly a method to kill hepatocellular carcinoma tissues through infections of liver tumor stem cells due to dengue viruses.

### Description of the Prior Art

Hepatoma second only to the lung cancer is the second most common cancer diagnosed in Taiwanese and one of top ten lethal diseases. The recurrence rate (70%) of hepatoma suffered by a liver cancer patient, who experienced a tumor excision and subsequent radioactive therapy or chemotherapeutics, remains high compared with other cancers. Amid lots of factors for recurrence of hepatoma, the cancer stem cell is the main reason. Cancer stem cells attributed to mutations of general stem cells and known for self-renewal and differentiation capacities are rare in all cancer cells (about 0.1%), probable mutating into a tumor again and revealing resistance to some drugs. Accordingly, cancer stem cells are not annihilated completely during chemotherapeutics but drive a cancer to relapse.

Against this background, how to provide a method with which cancer stem cells (also hereinafter referred to as “liver tumor stem cells”) in hepatocellular carcinoma tissues are infected preferentially and wiped out for least influence on normal hepatocytes, deaths of hepatocellular carcinoma tissues and no recurrence of hepatoma is the topic to be discussed herein.

According to previous researches, dengue viruses (DENV), which were found a potential to infect hematopoietic stem cells in marrows, are able to affect the hematopoietic system and damage the differentiation capacity of stem cells. As shown in literatures currently, antigens of dengue viruses have been detected in hepatocytes and Kupffer cells of a patient diagnosed with severe dengue fever and dengue viruses have been cultured in liver biopsies again. Moreover, it is asserted that the live is one of target organs infected by dengue viruses according to many clinical observations and mouse experiments. In addition, the fact that hepatoma cell lines are infected by dengue viruses for promotion of a cytopathic effect has been found by some researchers. However, there is still no evidence that liver tumor stem cells are infected by dengue viruses for deaths of liver tumor stem cells in literatures.

### SUMMARY OF THE INVENTION

Against the above background, the patent applicant deeply comprehending shortcomings and drawbacks in the prior art was devoted to an innovative design and successfully created a method to treat hepatoma with dengue viruses and a method to kill hepatocellular carcinoma tissues through dengue viruses after years of research.

To this end, the present disclosure provides a method to treat hepatoma with dengue viruses and a method to kill hepatocellular carcinoma tissues through infections of liver tumor stem cells due to dengue viruses.

In one embodiment of the present disclosure, hepatocellular carcinoma tissues include a completely cancerous tumor part as well as a non-tumor part between a normal liver and the cancerous tissues and the liver tumor stem cells are located in the tumor part.

In one embodiment of the present disclosure, infective dengue viruses are generated after infections of hepatocellular carcinoma tissues due to dengue viruses.

In one embodiment of the present disclosure, the viral titer of infective dengue viruses in a tumor part infected by dengue viruses is higher than that of infective dengue viruses in a non-tumor part infected by dengue viruses.

In one embodiment of the present disclosure, the viral titer of infective dengue viruses in liver tumor stem cells infected by dengue viruses is higher than that of infective dengue viruses in other non-liver tumor stem cells infected by dengue viruses.

In one embodiment of the present disclosure, biomarkers expressed by liver tumor stem cells are selected from at least one of a group consisting of CD133, CD117 and CD34.

In one embodiment of the present disclosure, biomarkers expressed by liver tumor stem cells comprise CD133.

In one embodiment of the present disclosure, liver tumor stem cells expressing the biomarker of CD133 in a tumor part are the objects infected by dengue viruses preferentially and killed due to specific protein expressions compared with cells expressing the biomarker of CD133 in a non-tumor part.

In one embodiment of the present disclosure, the ratio of the number of dengue viruses to the number of cells in a hepatocellular carcinoma tissue as the objects to be infected is between 0.5 MOI and 1.5 MOI.

In one embodiment of the present disclosure, the ratio of the number of dengue viruses to the number of cells in a hepatocellular carcinoma tissue as the objects to be infected is 1 MOI.

The present disclosure further provides a method to kill hepatocellular carcinoma tissues by dengue viruses which infect liver tumor stem cells in hepatocellular carcinoma tissues.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The techniques of present invention would be more understandable from the detailed description given herein below and the accompanying figures are provided for better illustration, and thus description and figures are not limitative for present invention, and wherein:

FIG. 1 is a flowchart for an experiment of hepatocellular carcinoma tissues infected by dengue viruses in one embodiment.

FIG. 2A, FIG. 2B and FIG. 2C illustrate test results of viral titers from hepatocellular carcinoma tissues infected by dengue viruses wherein FIG. 2A is the infection curves for tumor parts and non-tumor parts, both of which have been infected by dengue viruses (n=41), FIG. 2B displays comparison results of the peaks of titer for tumor parts and non-tumor parts, both of which have been infected by dengue viruses (n=41), and FIG. 2C shows correlations for the peaks of titer between a tumor part and a non-tumor part in each hepatocellular carcinoma tissue specimen (n=41).

FIG. 3A, FIG. 3B and FIG. 3C illustrate comparison results for peaks of titer with respect to different numbers of liver tumor stem cells (or non-liver tumor stem cells) after hepatocellular carcinoma tissues have been infected by dengue viruses in one embodiment wherein FIG. 3A is a scatter diagram for the peaks of titer with respect to different numbers of liver tumor stem cells (n=23) after tumor parts have been infected by dengue viruses, FIG. 3B is a scatter diagram for the peaks of titer with respect to the numbers of other cells (non-liver tumor stem cells) (n=23) after tumor parts have been infected by dengue viruses, and FIG. 3C shows percentages of gated cells expressing the biomarker of CD133 in tumor parts and non-tumor parts, respectively.

FIG. 4 illustrates test results for the biomarker of CD133 expressed in tumor parts infected by dengue viruses wherein blue line denotes an infection curve for a tumor part (i.e., cells intact and unsorted) infected by dengue viruses, the red line denotes an infection curve for a cell population expressing the biomarker of CD133 (CD133′ cells) after infection of a tumor part due to dengue viruses, and the green line denotes an infection curve for a cell population not expressing the biomarker of CD133 (CD133⁻ cells) after infection of a tumor part due to dengue viruses.

FIG. 5 illustrates the status of cell survival in a tumor part and a non-tumor part, each of which is still uninfected, after long-term culture wherein the red line and the blue line mean a cell survival curve (N=1) for long-term culture of cells in a tumor part and a cell survival curve (N=1) for long-term culture of cells in a non-tumor part, respectively.

FIG. 6A, FIG. 6B and FIG. 6C illustrate CD133⁺ cells in a tumor part as well as a non-tumor part and the significantly differential protein expressions in one embodiment wherein FIG. 6A, the volcano expression to compare protein expressions of CD133⁺ cells in a tumor part and a non-tumor part shows (1) the higher differential protein expressions in a tumor part are encircled with blue boxes at the right-hand side and summarized and ranked in a descending order of protein IDs in FIG. 6B and (2) the higher differential protein expressions in a non-tumor part are encircled with red boxes at the left-hand side and summarized and ranked in a descending order of protein IDs in FIG. 6C.

FIG. 7A to 7D illustrate lists of biological pathways participated by significantly differential protein expressions through analyses for the PANTHER database wherein significantly differential protein expressions are classified according to the molecular function as shown in FIG. 7A, differential protein expressions are classified according to the cellular component as shown in FIG. 7B, differential protein expressions are classified according to the biological process as shown in FIG. 7C, and differential protein expressions are classified according to the protein class as shown FIG. 7D.

FIG. 8A to 8B illustrate the ratios of biological pathways participated by cells with significantly differential protein expressions through analyses for the KEGG database wherein biological pathways participated by CD133′ cells with differential protein expressions in a tumor part are shown in FIG. 8A, biological pathways participated by CD133′ cells with differential protein expressions in a non-tumor part are shown in FIG. 8B, and the −log P value greater than 2 (P<0.05) is defined as a statistically significant difference.

### DETAILED DESCRIPTION OF THE INVENTION

### Terminology Definition

The technical and scientific terminologies common in the art of biotech are widely adopted in the patent specification; the definitions of these technical and scientific terminologies are given for clear and consistent understanding of the patent specification as well as claims hereinafter. Other terminologies not defined particularly hereinafter are well known to and commonly understood by persons skilled in the art.

The terminology of “individual”, “patient” or the like adopted herein means a mammal evaluated for any treatment and/or being treated. In one embodiment, a mammal is a human being. Accordingly, the terminology of “patient” means an individual diagnosed with hepatoma, an individual having experienced a tumor excision (operation) or a candidate to experience a tumor excision (operation). An individual can be a human being or another mammal species applicable to a lab model for human diseases, for example, mouse and rat.

The terminology of “treatment” or the like adopted herein means administration of a medicine for a certain effect on a patient. The effect denotes a certain disease and/or symptoms of the disease partially or completely cured through treatment. As shown in the present disclosure, the “treatment” means any cure of hepatoma diagnosed in a mammal (particularly a human being) and including (a) control of a disease, that is, progression of a disease inhibited and (b) a disease in remission, that is, symptoms relieved. For treatment of a tumor (for example, hepatoma), proliferation and metastasis of a tumor is alleviated by a therapeutic agent directly.

The terminology of “cell culture” or the like adopted herein means cells alive in ex vivo. The terminology of “cell culture”, however, should be interpreted as a general term for culture of a single cell, a tissue or an organ.

The terminology of “tumor” adopted herein means growths and proliferations of all neoplastic (benign or malignant), precancerous or cancerous cells and tissues.

The terminology of “hepatoma” adopted herein means phenomena for spontaneous and irregular growths of hepatocytes, that is, significantly uncontrollable cell proliferations and abnormal growth phenotypes. In the present disclosure, the cells to be detected, analyzed, classified or treated comprise precancerous (i.e., benign), malignant, pre-metastatic, metastatic and non-metastatic cells.

The terminology of “hepatocellular carcinoma tissue specimen” or the like adopted herein means a specimen in which all cells are collected from hepatocellular carcinoma tissues including a completely cancerous tumor part and a non-tumor part between the normal liver and cancerous tissues. In the case of a solid tumor without metastasis, a tissue specimen is collected from a tumor excised from a surgery and prepared and tested with a prior art. Similarly, in the case of a metastatic cancer, cells are collected from body fluids such as lymph, blood, serum or an infected distal organ and exudates thereof.

The terminology of “biomarker” adopted herein means specific bio-molecules (for example, CD133) significantly expressed by a specific cell population (for example, liver tumor stem cells).

The terminology of “recurrence of hepatoma” adopted herein means further proliferations of neoplastic or cancerous cells in a patient who was diagnosed with hepatoma. Specifically, the phenomenon of further proliferations of hepatocellular carcinoma tissues attributed to liver tumor stem cells which were not removed completely during treatment of hepatoma is called as recurrence of hepatoma.

The terminology of “with respect to”, “correlated with” or the like adopted herein means two events or cases with a statistical correlation between each other, for example, two numbers, data groups and the like. In the case of two numbers, for example, the positive correlation denotes a number increasing with another rising number and the negative correlation denotes a number decreasing with another rising number.

The terminology of “multiplicity of infection (MOI)” adopted herein means a ratio of the infective agent to an infection target, which refers to the formula, MOI=the number of viral particles/the number of host cells.

The terminology of “plaque forming unit (PFU)” adopted herein means the number of viruses for development of a viral plaque (bacteriophage plaque) on animal cells through monolayer culture and is the unit to quantify the content of viruses.

The terminology of “viral titer” adopted herein means the number of viruses in 1 ml culture medium that refers to the formula, viral titer (PFU/ml)=plaque unit*1000/400*(dilution factor).

[Dengue Virus]

Dengue viruses in the present disclosure show preference for infecting liver tumor stem cells. In the present disclosure, a serotype of dengue viruses is not limited to a specific category when hepatocellular carcinoma tissues are killed by dengue viruses that incline to infect liver tumor stem cells instead of normal hepatocytes mostly. As shown in one embodiment, a serotype of dengue viruses can be the dengue virus serotype 1 (DENV-1), the dengue virus serotype 2 (DENV-2), the dengue virus serotype 3 (DENV-3), the dengue virus serotype 4 (DENV-4), the dengue virus serotype 5 (DENV-5) or a virus serotype derived from one of above dengue virus serotypes. Dengue viruses can selected from one of above virus serotypes or a combination of two or more virus serotypes.

As shown in one embodiment, a serotype of dengue viruses is the dengue virus serotype 2 (DENV-2) preferably or the 16681 virus strain (strain 16681) of the dengue virus serotype 2 (DENV-2) optimally. With the source of dengue viruses not specifically designated in the present disclosure, dengue viruses are commercially available or extracted from a lab or a clinic test.

[Hepatic Stem Cells and Liver Tumor Stem Cells]

Hepatic stem cells, which have a potential to differentiate into hepatocytes, biliary epithelial cells and other types of cells, display biomarkers such as CD133, CD117 and CD34. Liver tumor stem cells attributed to mutations of hepatic stem cells and featuring self-renewal and differentiation capacities are the primary cell population infected by dengue viruses in the beginning. Liver tumor stem cells regarded as a minority in hepatoma cells (about 0.1%) can drive hepatoma cells to proliferate. Liver tumor stem cells as the objects infected by dengue viruses display at least one of biomarkers including CD133, CD117 and CD34 preferably or the biomarker of CD133 optimally. Liver tumor stem cells have developed resistance to some drugs which is one main reason of recurrence of hepatoma.

[Method to Treat Hepatoma]

A method to treat hepatoma with dengue viruses disclosed herein is to administer therapeutically effective dengue viruses, which prefer to infect and kill liver tumor stem cells, to a patient.

The method to treat hepatoma with dengue viruses disclosed herein is applicable to an individual mammal (human being particularly) which has come down with or risked a tumor as shown in the present disclosure.

The method to treat hepatoma with dengue viruses disclosed herein is to administer dengue viruses to an individual (for example, a sick human being) for inhibition of hepatoma cells. The method is also applicable to inhibiting proliferation of a patient's tumor in size, alleviating the disease burden of a tumor patient and/or improving clinical outcomes.

[Administration of Dengue Viruses]

The administration of dengue viruses to hepatocellular carcinoma tissues is effectuated through different pathways including intratumoral administration, intravenous injection, intracutaneous injection, subcutaneous injection, oral administration (for example, inhalation), percutaneous injection (i.e., topical injection), mucosal administration, intraperitoneal administration, intra-artery administration or rectal administration. Other applicable pathways for administration of a medical combination are effectuated through oral administration, transnasal administration, nasopharyngeal administration, parenteral administration, intestinal administration, gastric tube administration, topical injection, percutaneous injection, subcutaneous injection, intramuscular injection, pastille, solid, pulvis, liquid, aerosol, intralesional injection into a tumor, intralesional injection around a tumor, intravenous injection or arterial injection. Moreover, the topical or systemic administration in which excipients are added or not added is available; medications are administered into or around an individual's tumor by a slow release mode.

[Dosage]

The method to treat hepatoma with dengue viruses disclosed herein is to administer therapeutically effective dengue viruses to an individual as required. In detail, the administration depends on lots of factors: purpose of medicine administration; healthy conditions, age and category (for example, human being, non-human primate and primate) of an individual to be treated; formulation of dengue virus; medical conditions evaluated by a clinician; others. It is expected that a routine test is conducted through administration of dengue viruses with a dosage of relatively wide tolerance. For maximum deaths of liver tumor stem cells, the multiplicity of infection for dengue viruses is kept from 0.5 MOI to 1.5 MOI preferably or 1 optimally in contrast to the cell count of hepatocellular carcinoma tissues as the objects to be infected.

[Medical Combination]

A medical combination is prepared with dengue viruses and carriers pharmaceutically accepted (for example, salts pharmaceutically accepted) mixing each other; a medical combination is also packed in a vessel manufactured as a test kit or a product in which a package insert with information for applications of dengue viruses is included.

The present disclosure is demonstrated in embodiments as follows. The following embodiments, however, are examples of the present disclosure which are not considered as cases to restrict the present invention. Moreover, dengue viruses and materials used hereinafter are commercially available.

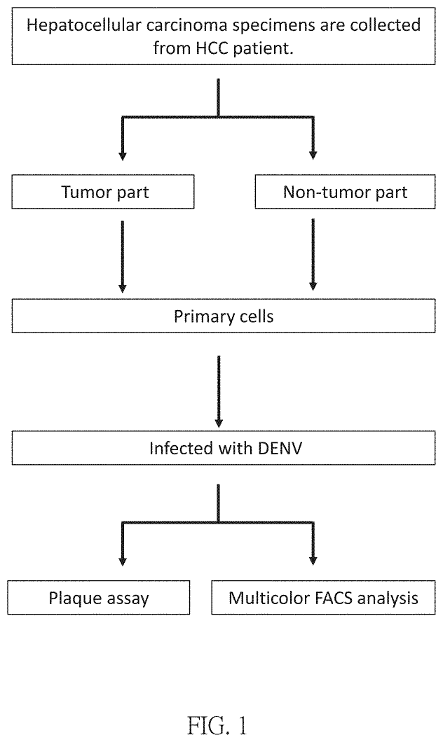

FIG. 1 is a flowchart for an experiment of hepatocellular carcinoma tissues infected by dengue viruses in one embodiment. As shown in FIG. 1, 41 hepatocellular carcinoma specimens are collected from patients diagnosed with hepatoma and each of 41 specimens includes a tumor part and a non-tumor part. The specimens collected are cut into pieces or ground and mixed with RBC (red blood cell) lysis buffers through which blood cells are removed but nucleated cells remain. Both a tumor part and a non-tumor part are infected with dengue viruses at 1MOI (the ratio of the number of viruses to the number of cells=1:1) and both infected supernates as well as cells are collected at specific points in time (Day 1, 2, 3, 5, 7, 10 and 14 after infection): the viral titer is quantified in a plaque assay for the supernates; the cell surface markers are analyzed by the multicolor FACS analysis.

[Hepatocellular Carcinoma Tissue Specimens]

Hepatocellular carcinoma tissue specimens are collected from 41 tumor patients (Project No. B-ER-103-187 of the Institutional Review Board, National Cheng Kung University Hospital) and genders as well as ages of patients as the source of specimens are summarized in Table 1. Each specimen includes paired parts, i.e., tumor part and non-tumor part. A specimen newly collected is cut into pieces and soaked in PBS first and mixed with 2-3 ml collagenase for 5-minute culture at 37 degrees Celsius and removals of connective tissues. The enzyme reaction is neutralized by the RPMI medium with 10% fetal bovine serums. The primary cells are separated by a centrifuge. Next, RBC lysis buffers are added into the specimen inside a rotator for an 8-minute reaction and the specimen is transferred to a centrifuge (300 rcf; 8 minutes) for removals of red blood cells (this step should be repeated twice for complete removals of red blood cells). The cells in the specimen are re-dissolved in the RPMI medium with 10% fetal bovine serums and screened out by 100 μm filter membranes for removals of coagulated cells and calculation of cell counts for following tests.

TABLE 1
Patients with HCC specimens (n = 41)

| Group | Amount |
|---|---|
| Gender | |
| Male | 32 |
| Female | 9 |
| Age | |
| 30-50 | 7 |
| 51-70 | 24 |
| 71-84 | 10 |

[Plaque Assay]

BHK (baby hamster kidney) cells (7×10⁵/well) are dissolved in 1.5 DMEM medium containing 5% fetal bovine serums and planted on a 6-well plate. As a determinand, virus fluids processed in serial dilution are prepared with 100 μL unknown virus fluids added into 900 μL DMEM medium containing 2% fetal bovine serums for 10-fold dilution. With cells adhering to a 6-well plate, the culture fluid is removed and 400 μL virus fluids to be tested are added for 2-hour culture in an incubator at 37 degrees Celsius. The 6-well plate is shaken once every 15 minutes for infected fluids uniformly covering cells. The infected fluids are removed two hours later and methylcellulose is added into the 6-well plate in which viruses move within a restricted region for 7-day culture in an incubator at 37 degrees Celsius. The 6-well plate is taken out from the incubator 7 days later and cleaned with PBS applied in all wells. Except an area on which no cell exists or cells are dead, cells are stained by crystal violet for one hour. With stains rinsed by fresh water, the number of viral plaques developed is estimated for calculation of a viral titer.

[Ex Vivo Infection of DENV]

The ex vivo infections due to dengue viruses are made with 8×10⁶ tumor part cells and 8×10⁶ non-tumor part cells dissolved in RPMI medium which contains 10% fetal bovine serums (infection condition: 1MOI; total volume of virus and cell fluids: 2 ml; serotype of dengue viruses: DENV-2 (strain 16681)), respectively. The infected mixture fluids are cultured in an incubator at 37 degrees Celsius for two hours and the mixture fluid tubes are shaken every 30 minutes for full contacts between viruses and cells. Viruses not contacting cells are removed by a centrifuge (300rcf; 8 minutes) two hours later; cells are re-dissolved by 700 μL RPMI medium containing 10% fetal bovine serums and dispensed to seven centrifuge tubes, each of which contains RPMI medium with 10% fetal bovine serums (2 ml) and placed in an incubator at 37 degrees Celsius for collections of virus fluids as well as cell fluids at specific points in time (Day 1, 2, 3, 5, 7, 10 and 14 after infection). The infected mixture fluids are taken out from the incubator at the specific time and placed in a centrifuge (300rcf; 8 minutes) for separation between supernates and cells and inspection of a viral titer in the plaque assay for infected supernates. The cells are rinsed with PBS for following tests.

[Magnetic Beads Isolation]

5×10⁷ tumor part cells (non-tumor part cells) are rinsed with MACS buffers for removals of residual cell fluids; cells are re-dissolved by appropriate MACS buffers and added by magnetic beads which linked specific antibodies. It is necessary for every 10⁷ cells to mix with both 20 μL magnetic beads linking specific antibodies and 80 μL MACS buffers. The fluids in which cells and magnetic beads are mixed are placed on a rotary device and stored in a refrigerator for a 40-minute reaction at 4 degrees Celsius. After the reaction, the mixture fluids are added by 2-3 ml MACS buffers and transmitted to a centrifuge (1000 rpm; 5 minutes) for separation between cells and magnetic beads not linking cells. Cells are re-dissolved by 500 μL MACS buffers and added into an LS column which has been installed on a magnetic base and rinsed with 1 ml MACS buffer in advance. In this step, cells linking magnetic beads and staying in the LS column and cells not linking magnetic beads and guided into a centrifugal tube from the LS column are classified as positive cells and negative cells, respectively. Moreover, the LS column is rinsed with MACS buffers such that all cells staying in the LS column are positive cells. Finally, with the LS column removed from the magnetic base, cells inside the LS column are transmitted to a new centrifugal tube. Each of the two centrifugal tubes in which cells are accommodated is placed on a centrifuge (300 rpm; 8 minutes) for removals of MACS buffers; cells are re-dissolved by RPMI medium containing 10% fetal bovine serums for calculation of cell counts.

[Multicolor FACS Analysis]

2×10⁶ cells are re-dissolved by 200 μL staining buffers and separated into the staining group and the isotype group. Immunofluorescent antibodies for a specific cell surface marker are added into cells of the staining group and cells of the isotype group for staining, respectively. Cells should be kept away from light rays during a staining step and stored in a refrigerator for a 30-minute reaction at 4 degrees Celsius. After the reaction, cells are rinsed with 1 ml staining buffer and transmitted to a centrifuge (300 rcf; 8 minutes) for removals of antibodies not linking cells. Finally, cells are re-dissolved by 200 μL staining buffers and cell surface markers expressed are checked with a BD LSRFortessa™ cell analyzer.

### Embodiment 1

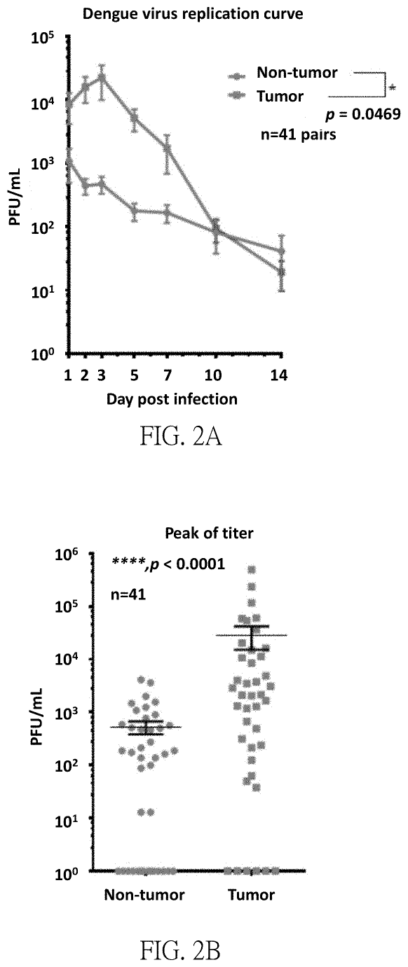

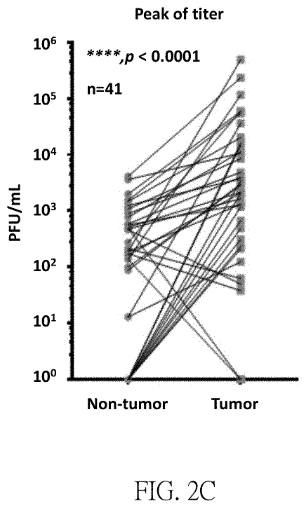

Referring to FIG. 2A, FIG. 2B and FIG. 2C, which illustrate test results of viral titers from hepatocellular carcinoma tissues infected by dengue viruses in the embodiment. FIG. 2A are infection curves for tumor parts and non-tumor parts, both of which have been infected by dengue viruses (n=41). It can be seen from test results of viral titers that hepatocellular carcinoma tissues are infected by dengue viruses and infective virus are generated. In FIG. 2A, FIG. 2B and FIG. 2C, circle dots and squares constitute infection curves of non-tumor parts and infected tumor parts, respectively; the statistical difference exists between two curves (*, p=0.0469). That is, dengue viruses prefer to infect a tumor part and proliferate dramatically.

Furthermore, the peaks of titer for a tumor part and a non-tumor part, both of which have been infected by dengue viruses, in each specimen are analyzed. As shown in FIG. 2B, the peaks of titer for tumor parts and non-tumor parts, both of which have been infected by dengue viruses, are compared (n=41). It can be seen from test results that the viral titer of a tumor part infected by dengue viruses is significantly higher than that of a non-tumor part infected by dengue viruses and the statistical difference exists (****, p<0.0001). That is, dengue viruses prefer to infect a tumor part and proliferate dramatically.

In addition, the correlation of viral titers between a tumor part and a non-tumor part, both of which have been infected by dengue virus, in each specimen is analyzed. The correlation for the peaks of titer between a tumor part and a non-tumor part in each hepatocellular carcinoma tissue specimen is shown in FIG. 2C (n=41). It can be seen from test results that the viral titer of a tumor part infected by dengue viruses is higher than the viral titer of a non-tumor part infected by dengue viruses. That is, dengue viruses prefer to infect a tumor part and proliferate dramatically.

### Embodiment 2

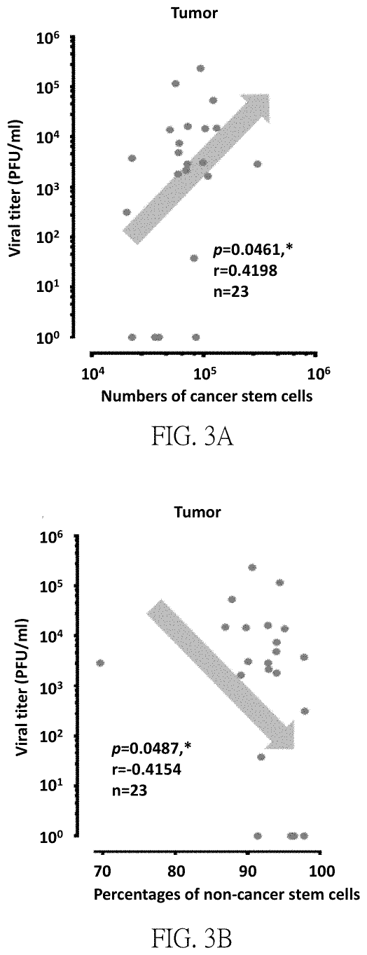

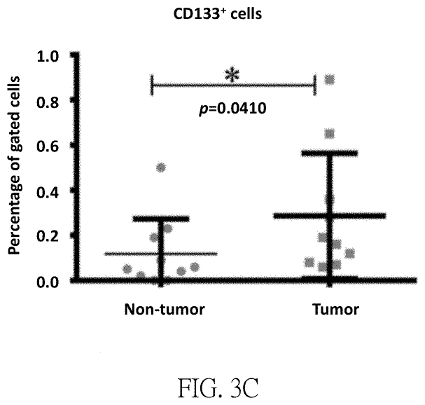

Referring to FIG. 3A, FIG. 3B and FIG. 3C, which illustrate comparison results for peaks of titer with respect to different numbers of liver tumor stem cells (or non-liver tumor stem cells) after hepatocellular carcinoma tissues have been infected by dengue viruses in the embodiment. The peaks of titer with respect to different numbers of liver tumor stem cells are shown in a scatter diagram in FIG. 3A (n=23) after tumor parts have been infected by dengue viruses. It can be seen from stained cells that a significant positive correlation (r>0) exists between a peak of titer for hepatocellular carcinoma tissues infected by dengue viruses and the number of liver tumor stem cells (CD133^{+/-}, CD117^{+/-} and/or CD34^{+/-} cells) in hepatocellular carcinoma tissues and a statistical difference is observed (*, p=0.0461). That is, dengue viruses prefer to infect liver tumor stem cells and proliferate dramatically.

On the contrary, the peaks of titer with respect to the numbers of other non-liver tumor stem cells are shown in a scatter diagram in FIG. 3B (n=23) after tumor parts have been infected by dengue viruses. It can be seen from stained cells that a significant negative correlation (r<0) exists between a peak of titer for hepatocellular carcinoma tissues infected by dengue viruses and the number of other non-liver tumor stem cells in hepatocellular carcinoma tissues and a statistical different is observed (*, p=0.0487). That is, dengue viruses prefer not to infest other non-liver tumor stem cells and proliferate moderately.

Then, the expressions of a biomarker for liver tumor stem cells in a tumor part and a non-tumor part are analyzed. FIG. 3C illustrates percentages of gated cells expressing the biomarker of CD133 in tumor parts and non-parts, respectively. It can be seen from comparison results that a significant difference is observed in the biomarker of CD133 between tumor parts and non-tumor parts. That is, there are more liver tumor stem cells (cells expressing CD133) inside tumor parts than those inside non-tumor parts.

As mentioned in the previous section, the viral titer developed from a tumor part infected by dengue viruses is significantly correlated with the number of liver tumor stem cells in hepatocellular carcinoma tissues and the cell population expressing CD133⁺ (CD133⁺ cells) in a tumor part is significantly different from the cell population expressing CD133⁺ (CD133⁺ cells) in a non-tumor part.

### Embodiment 3

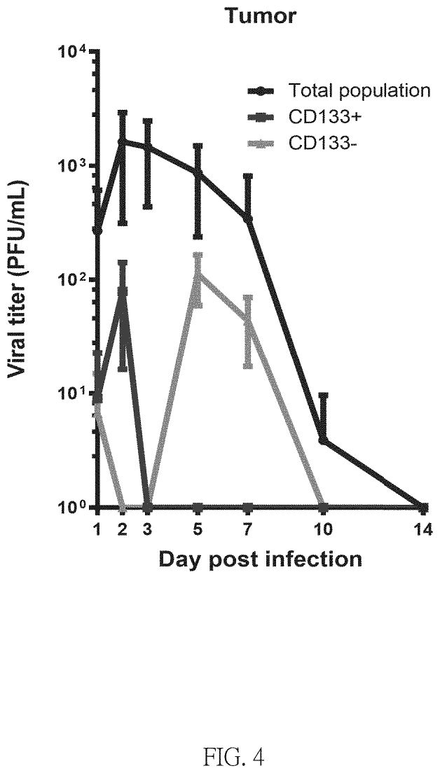

Referring to FIG. 4, which illustrates test results for the biomarker of CD133 expressed in tumor parts infected by dengue viruses in the embodiment. With cells expressing CD133⁺ (CD133⁺ cells) and cells expressing CD133⁻ (CD133⁻ cells) separated from a tumor part by the magnetic cell separation technology, these cells are infected by dengue viruses and infected supernates are collected at specific points in time for quantification of viruses based on the plaque assay. As shown in FIG. 4, the blue line denotes an infection curve for a tumor part (i.e., cells intact and unsorted) infected by dengue viruses, the red line denotes an infection curve for a cell population expressing the biomarker of CD133 (CD133⁺ cells) after infection of a tumor part due to dengue viruses, and the green line denotes an infection curve for a cell population not expressing the biomarker of CD133 (CD133⁻ cells) after infection of a tumor part due to dengue viruses. It can be seen from test results that (1) preliminary stage (from Day 1 to Day 3) after infections of cells due to dengue viruses: CD133⁺ cells are dominant for proliferations of infective viruses; (2) middle or later stage (three days later) after infections of cells due to dengue viruses: CD133⁻ cells are dominant for proliferations of infective viruses.

### Embodiment 4

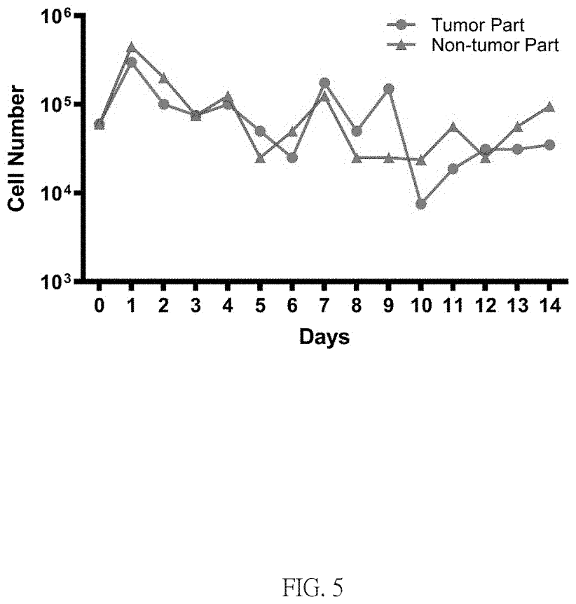

Referring to FIG. 5 that illustrates the status of cell survival in a tumor part and a non-tumor part, each of which is still uninfected, after long-term culture in the embodiment; that is, 6×10⁴ initiating cells are cultured in a tumor part (a non-culture part) at 37 degrees Celsius from Day 1 to Day 14 during which the cell count in each of both parts is estimated every day. As shown in FIG. 5, the red line and the blue line mean a cell survival curve (N=1) for long-term culture of cells in a tumor part and a cell survival curve (N=1) for long-term culture of cells in a non-tumor part, respectively. It can be seen from test results that two cell survival rates in both a tumor part and a non-tumor part, each of which is still uninfected, are similar to each other, that is, both cells survive at least 14 days.

In summary, for no significant difference in cell counts between a tumor part and a non-tumor part within 14 days, a non-tumor part is less likely than a tumor part to be infected by dengue viruses. Despite a specific reason unknown, it can be inferred that liver tumor stem cells, particularly liver tumor stem cells expressing CD133⁺, are the main objects infected by dengue viruses and fewer liver tumor stem cells existing in a non-tumor part survive infections. In fact, liver tumor stem cells expressing CD133′ should not be found inside a non-tumor part, which exists between normal hepatic tissues and cancerous tissues, theoretically; however, detected in a non-tumor part, liver tumor stem cells expressing CD133⁺ are considered as metastatic cells from a tumor part presumptively and cause infection of a non-tumor part by dengue viruses.

### Embodiment 5

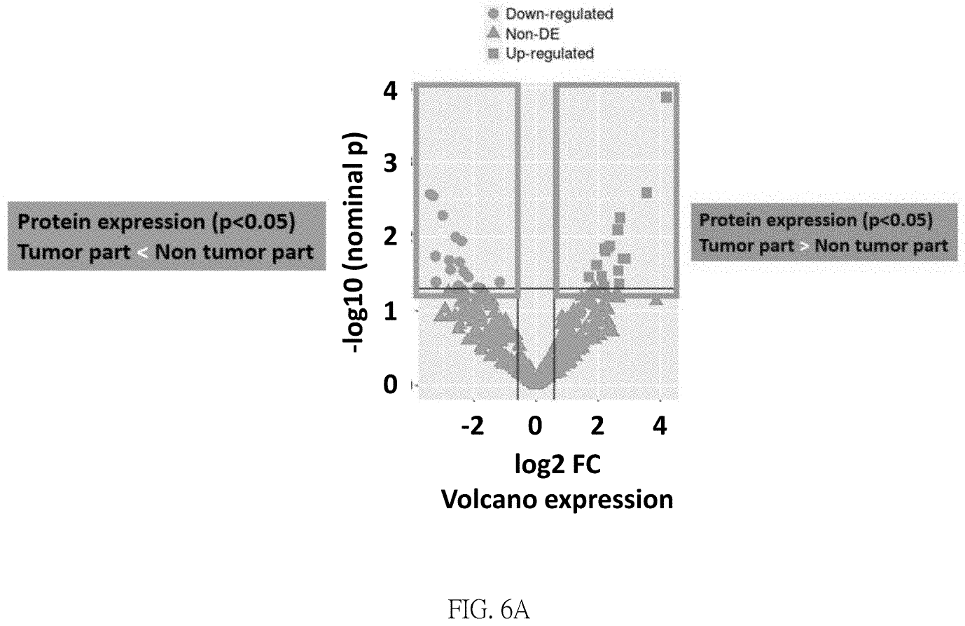

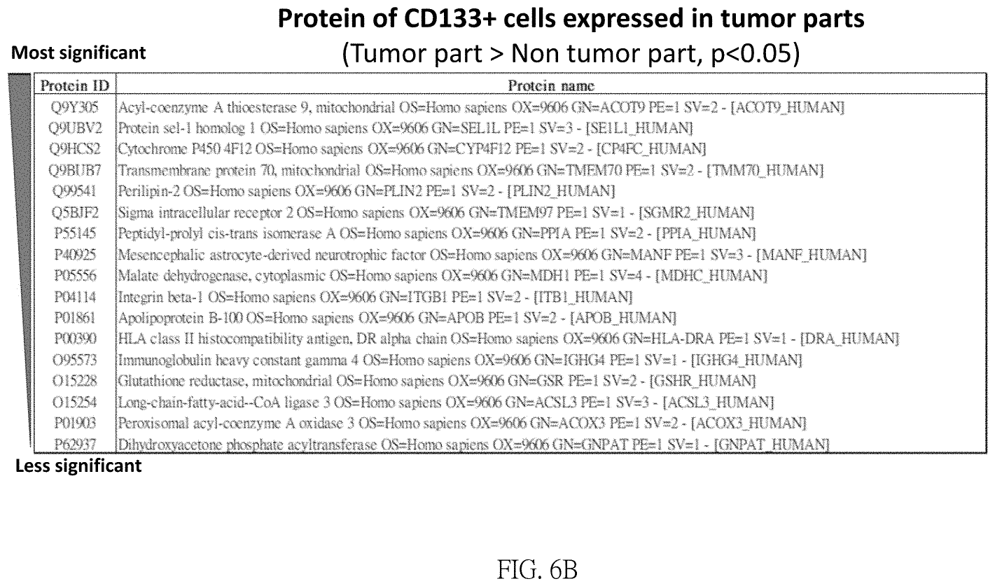

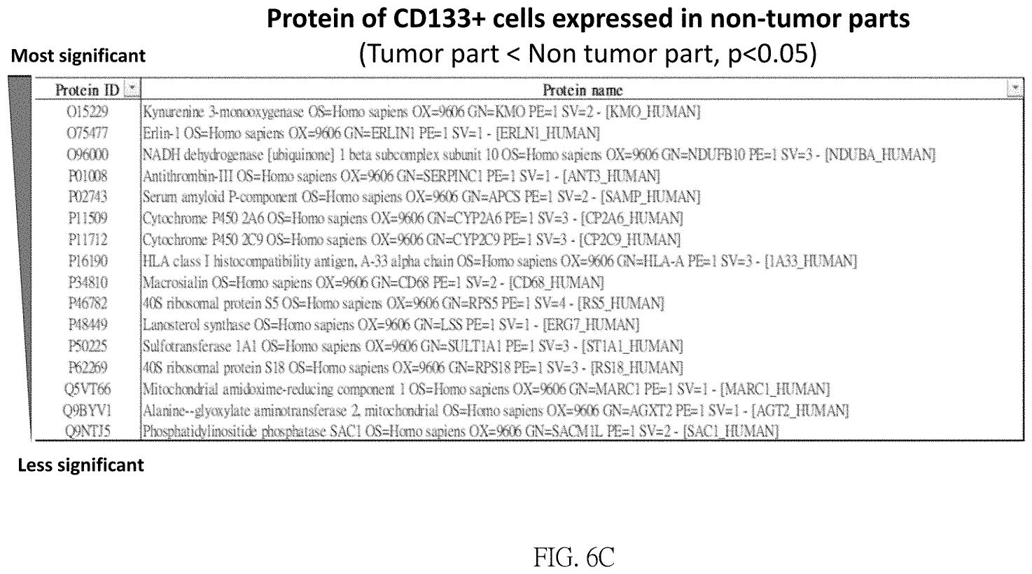

FIG. 6A, FIG. 6B and FIG. 6C illustrate CD133⁺ cells expressed in a tumor part as well as a non-tumor part and the significantly differential protein expression in the embodiment. The protein expressions of CD133⁺ cells, which are extracted from a tumor part and a non-tumor part, respectively, are analyzed with the proteomics analysis. Any significantly differential protein expression in CD133⁺ cells between a tumor part and a non-tumor part is discovered from initial test results by DE analysis App for the difference analysis, as shown in FIG. 6A for the volcano expression. It can be seen from protein expressions of CD133⁺ cells in a tumor part and a non-tumor part that (1) the higher differential protein expressions in a tumor part are encircled with blue boxes at the right-hand side and summarized and ranked in the descending order of protein IDs in FIG. 6B, that is, Q9Y305, Q9UBV2, Q9HCS2, Q9BUB7, Q99541, Q5BJF2, P55145, P40925, P05556, P04114, P01861, P00390, 095573, 015228, 015254, P01903 and P62937; (2) the higher differential protein expressions in a non-tumor part are encircled with red boxes at the left-hand side and summarized and ranked in the descending order of protein IDs in FIG. 6C, that is, 015229, 075477, 096000, P01008, P02743, P11509, P11712, P16190, P34810, P46782, P48449, P50225, P62269, Q5VT66, Q9BYV1 and Q9NTJ5.

As shown in above results, the protein IDs of liver tumor stem cells expressing the biomarker of CD133 in a tumor part are Q9Y305, Q9UBV2, Q9HCS2, Q9BUB7, Q99541, Q5BJF2, P55145, P40925, P05556, P04114, P01861, P00390, 095573, 015228, 015254, P01903 and P62937, each of which has a raised protein expression, and the protein IDs of liver tumor stem cells expressing the biomarker of CD133 in a non-tumor part are 015229, 075477, 096000, P01008, P02743, P11509, P11712, P16190, P34810, P46782, P48449, P50225, P62269, Q5VT66, Q9BYV1 and Q9NTJ5, each of which has a lower protein expression. Accordingly, liver tumor stem cells expressing the biomarker of CD133 inside a tumor part are objects infected by dengue viruses preferentially and killed consequentially.

### Embodiment 6

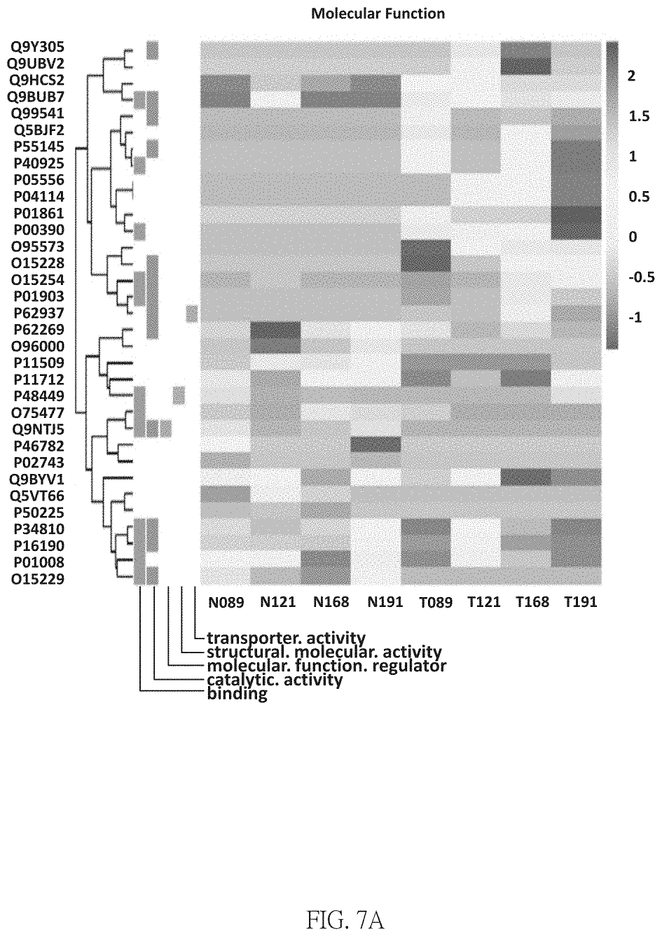

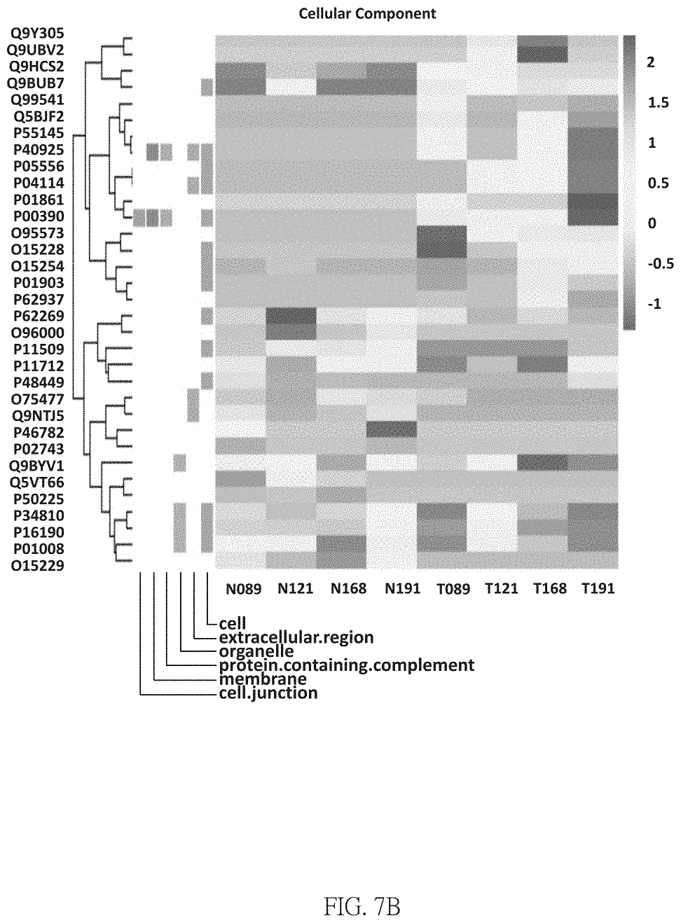

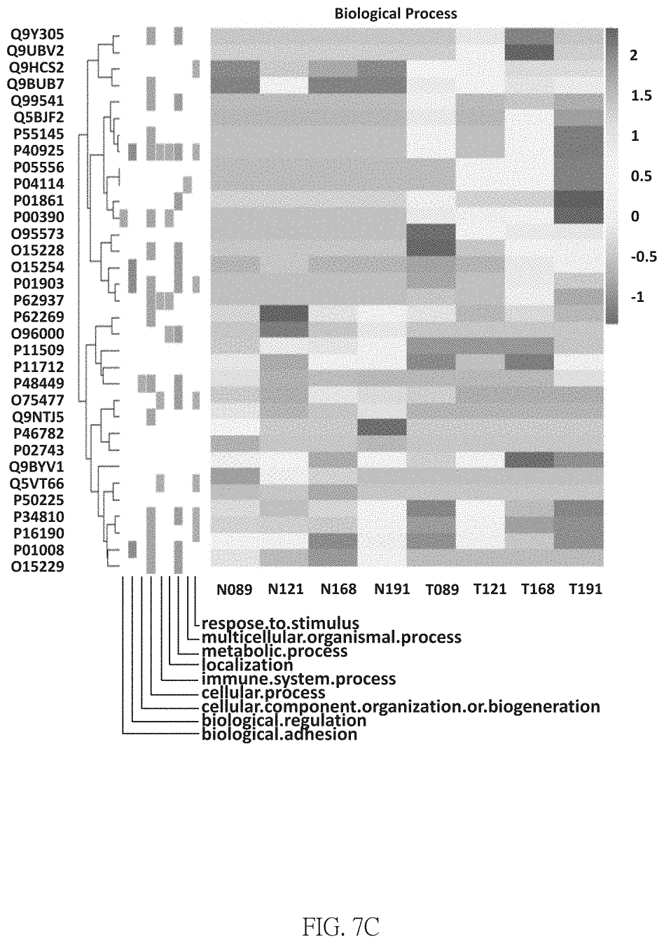

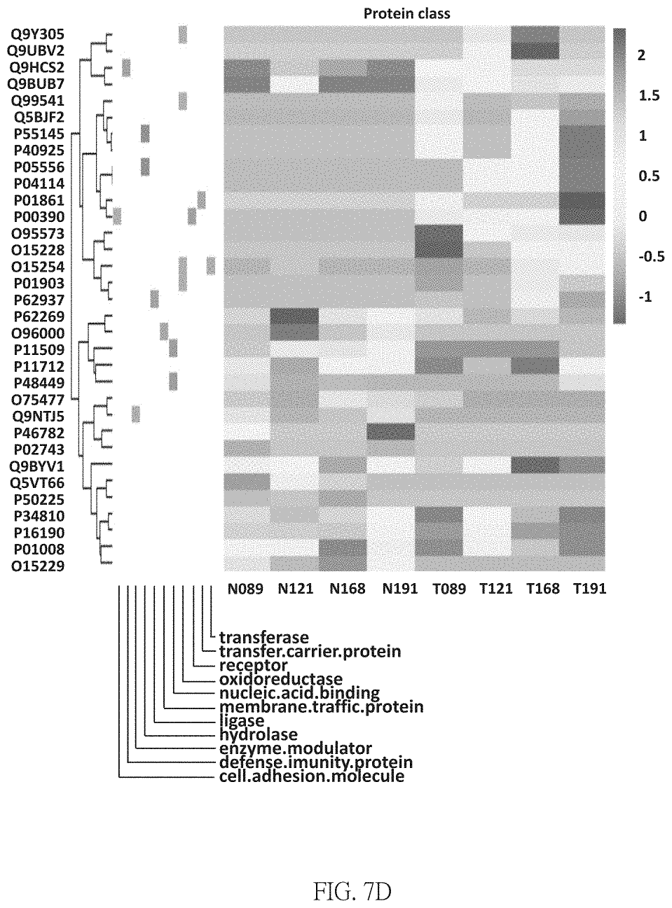

Referring to FIG. 7A to 7D, which illustrate lists of biological pathways participated by significantly differential protein expressions through analyses for the PANTHER database in the embodiment. The physiological functions and the biological pathways participated by significantly differential protein expressions as shown in Embodiment 4 are analyzed based on the PANTHER database and these proteins are classified according to molecular function, cellular component, biological process and protein class, respectively. FIG. 7A illustrates significantly differential protein expressions are classified according to the molecular function. It can be seen from FIG. 7A that the molecular functions including transporter activity, structural molecular activity, molecular function regulator, catalytic activity and binding in CD133⁺ cells between a tumor part and a non-tumor part are affected. FIG. 7B illustrates differential protein expressions are classified according to the cellular component. It can be seen from FIG. 7B that the cellular components including cell, extracellular region, organelle, protein containing complex, membrane and cell junction in CD133⁺ cells between a tumor part and a non-tumor part are affected. FIG. 7C illustrates differential protein expressions are classified according to the biological process. It can be seen from FIG. 7C that the biological processes including response to stimulus, multicellular organismal process, metabolic process, localization, immune system process, cellular process, cellular component organization or biogene, biological regulation and biological adhesion in CD133⁺ cells between a tumor part and a non-tumor part are affected. FIG. 7D illustrates differential protein expressions are classified according to the protein class. It can be seen from FIG. 7D that the protein classes including transferase, transfer carrier protein, receptor, oxidoreductase, nucleic acid binding, membrane traffic protein, ligase, hydrolase, enzyme modulator, defense immunity protein and cell adhesion molecule in CD133⁺ cells between a tumor part and a non-tumor part are affected.

As illustrated in previous results, the molecular function, the cellular component, the biological process and the protein class for liver tumor stem cells expressing the biomarker of CD133 in a tumor part are affected. Accordingly, liver tumor stem cells expressing the biomarker of CD133 in a tumor part are objects infected by dengue viruses preferentially and killed consequentially.

### Embodiment 7

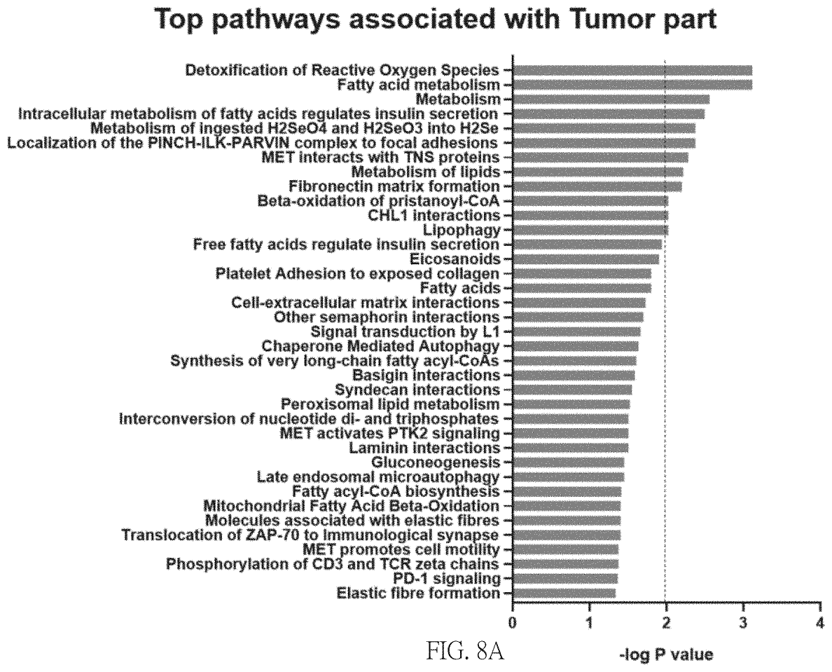

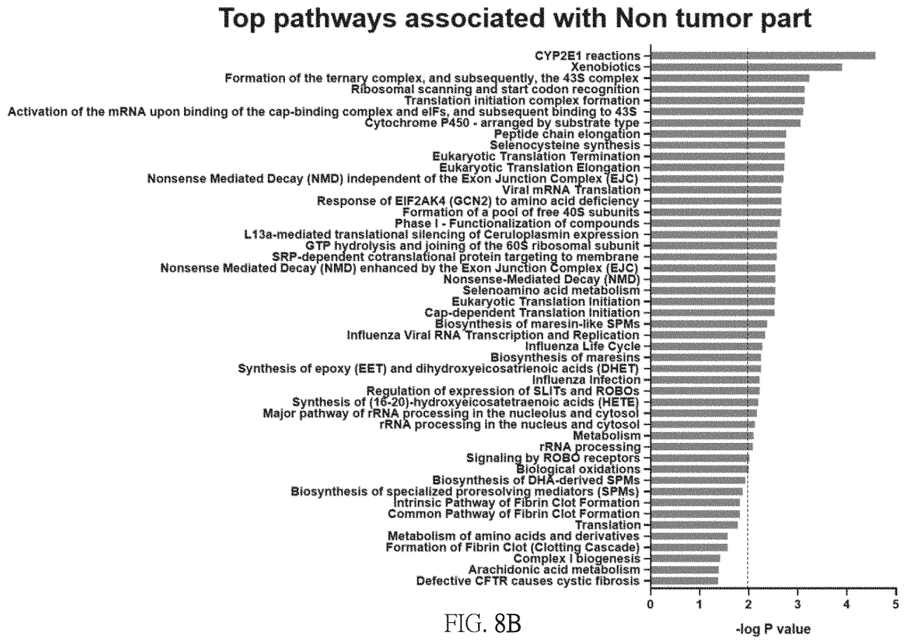

Referring to FIGS. 8A and 8B, which illustrate the ratios of biological pathways participated by significantly differential protein expressions through analyses for the KEGG database in the embodiment. As shown in FIGS. 8A and 8B, the −log P value greater than 2 (P<0.05) is defined as a statistically significant difference. FIG. 8A illustrates these biological pathways participated by CD133′ cells with differential protein expressions in a tumor part are ranked in the descending order of significant differences and shown as follows: detoxification of reactive oxygen species, fatty acid metabolism, metabolism, intracellular metabolism of fatty acids regulates insulin secretion, metabolism of ingested H₂SeO₄ and H₂SeO₃ into H₂Se, localization of the PINCH-ILK-PARVIN complex to focal adhesions, MET interacts with TNS proteins, metabolism of lipids, fibronectin matrix formation, beta-oxidation of pristanoyl-CoA, CHL1 interactions and lipophagy; FIG. 8B illustrates these biological pathways participated by CD133′ cells with differential protein expressions in a non-tumor part are ranked in the descending order of significant differences and shown as follows: CYP2E1 reactions, Xenobiotics, formation of the ternary complex and subsequently the 43S complex, ribosomal scanning and start codon recognition, translation initiation complex formation, activation of the mRNA upon binding of the cap-binding complex and elFs and subsequent binding to 43S, cytochrome P450 arranged by substrate type, peptide chain elongation, selenocysteine synthesis, eukaryotic translation termination, eukaryotic translation elongation, Nonsense Mediated Decay (NMD) independent of the Exon Junction Complex (EJC), viral mRNA translation, response of ElF2AK4 (GCN2) to amino acid deficiency, formation of a pool of free 40S subunits, phase I functionalization of compounds, L13a-mediated translational silencing of ceruloplasmin expression, GTP hydrolysis and joining of the 60S ribosomal subunit, SRP-dependent cotranslational protein targeting to membrane, Nonsense Medited Decay (NMD) enhanced by the Exon Junction Complex (EJC), Nonsense-Mediated Decay (NMD), selenoamino acid metabolism, eukaryotic translation initiation, cap-dependent translation initiation, biosynthesis of maresin-like SPMs, influenza viral RNA transcription and replication, influenza life cycle, biosynthesis of maresins, synthesis of epoxy (EET) and dihydroxyeicosatrienoic acids (DHET), influenza infection, regulation of expression of SLITs and ROBOs, synthesis of (16-20)-hydroxyeicosatetraenoic acids (HETE), major pathway of rRNA processing in the nucleolus and cytosol, rRNA processing in the nucleus and cytosol, metabolism, rRNA processing, signaling by ROBO receptors and biological oxidations.

Because these biological pathways for liver tumor stem cells expressing the biomarker of CD133 in a tumor part are affected as shown in above test results, the liver tumor stem cells expressing the biomarker of CD133 in a tumor part are the objects infected by dengue viruses preferentially and killed consequentially.

In summary, more and more infective dengue viruses are generated in a tumor part of hepatocellular carcinoma tissues infected by dengue viruses; the viral titer of generated dengue viruses changes proportionally with the percentage of liver tumor stem cells (CD133⁺ cells), which have been infected by dengue viruses, in a rumor part and a significantly positive correlation is observed between the viral titer of dengue viruses and the number of liver tumor stem cells. Therefore, liver tumor stem cells are infected and killed by dengue viruses.

It is inferred that one reason for infections of liver tumor stem cells (CD133′ cells) in a tumor part due to dengue viruses preferentially is the higher number of liver tumor stem cells expressing the biomarker of CD133 in a tumor part than the number of cells expressing the biomarker of CD133 in a non-tumor part; moreover, due to influence on specific protein expressions (as shown in FIG. 6A, FIG. 6B and FIG. 6C), liver tumor stem cells expressing the biomarker of CD133 in a tumor part become the objects infected by dengue viruses preferentially and killed consequentially.

The above embodiments used to explain technical philosophy and characteristics in the present invention are aimed at the present disclosure understood and put into practice by persons skilled in the art but not considered as restrictions to claims hereinafter, that is, any equivalent change or modification based on spirit of the preset disclosure should be included in claims hereinafter.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method to treat hepatoma with dengue viruses, wherein hepatocellular carcinoma tissues are killed when liver tumor stem cells are infected by the dengue viruses.

2. The method to treat hepatoma with dengue viruses as claimed in claim 1, wherein the hepatocellular carcinoma tissues comprise a completely cancerous tumor part and a non-tumor part between a normal liver and cancerous tissues and the liver tumor stem cells are located in the tumor part.

3. The method to treat hepatoma with dengue viruses as claimed in claim 2, wherein infective dengue viruses are generated with the hepatocellular carcinoma tissues infected by the dengue viruses.

4. The method to treat hepatoma with dengue viruses as claimed in claim 3, wherein a viral titer of the infective dengue viruses in the tumor part infected by the dengue viruses is higher than that of the infective dengue viruses in the non-tumor part infected by dengue viruses.

5. The method to treat hepatoma with dengue viruses as claimed in claim 3, wherein a viral titer of the infective dengue viruses in the liver tumor stem cells infected by the dengue viruses is higher than that of the infective dengue viruses in other non-liver tumor stem cells infected by the dengue viruses.

6. The method to treat hepatoma with dengue viruses as claimed in claim 1, wherein biomarkers expressed by the liver tumor stem cells are selected from at least one of a group consisting of CD133, CD117 and CD34.

7. The method to treat hepatoma with dengue viruses as claimed in claim 6, wherein the biomarkers expressed by the liver tumor stem cells comprise CD133.

8. The method to treat hepatoma with dengue viruses as claimed in claim 6, wherein the liver tumor stem cells expressing the biomarker of CD133 in the tumor part are objects infected by the dengue viruses preferentially and further killed due to specific protein expressions compared with cells expressing the biomarker of CD133 in the non-tumor part.

9. The method to treat hepatoma with dengue viruses as claimed in claim 1, wherein a ratio of a number of the dengue viruses to a number of cells in a hepatocellular carcinoma tissue of an object to be infected ranges from 0.5 MOI to 1.5 MOI.

10. The method to treat hepatoma with dengue viruses as claimed in claim 1, wherein a ratio of a number of the dengue viruses to a number of cells in a hepatocellular carcinoma tissue of an object to be infected is 1 MOI.

11. A method to kill hepatocellular carcinoma tissues by dengue viruses which infect liver tumor stem cells in hepatocellular carcinoma tissues for annihilation of the hepatocellular carcinoma tissues.
